(19) United States
(12) Patent Application Publication
(10) Pub. No.: US 2014/0046235 A1
(43) Pub. Date: Feb. 13, 2014

(54) Dynamic Load Bearing Shock Absorbing Exoskeletal Knee Brace

(71) Applicant: Egas Jose-Joaquim DeSousa, Grand Blanc, MI (US)

(72) Inventor: Egas Jose-Joaquim DeSousa, Grand Blanc, MI (US)

(21) Appl. No.: 13/902,835

(22) Filed: May 26, 2013

Related U.S. Application Data
(63) Continuation-in-part of application No. 13/555,165, filed on Aug. 9, 2012.

(51) Int. Cl. A61F 5/01

(52) U.S. Cl. CPC A61F 5/0123
USPC 602/16

(57) ABSTRACT

The exoskeletal dynamic load bearing shock absorbing knee brace makes use of the energy absorbing characteristics of specifically designed industrial shock absorbers which are held precisely in place by an articulated dynamic exoskeletal structure that is designed not to restrict any freedom of movement when attached to the legs of the individual with the injured knee using the device. The exoskeletal structure is designed using the principles of the overcenter linkage to translate and transfer precisely and accurately in a repeatable manner, to the shock absorbers a representative fraction of the normal and extra normal ambulatory movements of the lower limbs of the user which makes possible for these shock absorbers to absorb a corresponding amount of energy, and provide an alternate load bearing structure parallel to the knees thus introducing a desirable degree of protection for the injured knee.

This is a continuation in part of the non provisional application Ser. No. 13/555,165 with filing date of Aug. 9, 2012 confirmation number 6657.

### FIELD OF THE INVENTION

This invention is in the field of orthotics. Specifically it provides for a knee orthotic device incorporating external shock absorbers appropriately designed to supplement the load bearing and shock absorbing function of the impaired meniscus in the knee joint. This continuation in part further provides for a refined design of the knee support device that allows the individual wearing it to retain the complete freedom of movement of the lower limb and the foot along all the axes. This design also continues to enable the afflicted individual wearing the device to experience a greater degree of comfort as the external shock absorbers are actuated at heel strike.

### BACKGROUND OF THE INVENTION

Injuries to the human knee joint are all too common resulting from participation in extreme sporting activities or resulting from accumulated damage with advancing years.

The knee joint is the biggest joint in the human body and is subject to various failure modes. One of the failure modes that this present invention addresses is the failure of the meniscus to maintain its integrity under sudden load spikes or accumulated damage. The meniscus is an avascular cartilage that acts as a shock absorber inside the knee. There are two disc shaped menisci in each knee.

FIG. 1 shows a representation of the knee with the meniscus between the Femur and the Tibia.

The damage resulting from meniscus tears and other failure modes is hard to contain and control under normal everyday working load situations. As the meniscus failure progresses the Femur and the Tibia come in direct contact resulting in irreversible damage under very painful conditions. Eventually, the afflicted individual in most cases, unable to bear the pain, will ask for and receive artificial knees depending upon the circumstances.

The present invention provides a load bearing shock absorbing device that could have a role in preventing further damage and help in the healing of the damaged knee joint. It could also be used to enable, otherwise impaired individuals to continue on with their active prior lifestyle

A Sep. 11, 2008 study in the New England Journal of Medicine titled Incidental Meniscal Findings on Knee MRI in Middle-Aged and Elderly Persons by seven physicians in the New England area concluded that “Incidental Meniscal findings on MRI of the knee are common in the general population and increase with increasing age.” The study reports Meniscal tear prevalence rates of 15% to 30% in women and men aged 50-59 and 27% to 37% rates in women and men aged 60-69. The incidence rates increase to a maximum of 50% with age advancing to 70-90 years.

A lot of these people learn to live with the pain. It is one of the objectives of this invention to help ameliorate the painful conditions.

Brief Overview Of The Prior Art

Most knee braces and supporting orthotics currently available in the marketplace are not targeted at providing the load bearing, shock absorbing function that is the key feature of the present invention.

A comprehensive search of available knee braces uncovers many very well built devices meant to support the injured knees. But these knee braces do not provide the load bearing and shock absorbing function of the present device.

A Patent search revealed the following prior art that is somewhat relevant to the present invention:
1. U.S. Pat. No. 4,688,599 Vito et al. This one is designed to provide stability in cases of loss of neuro-muscular control of a knee joint or a hip joint.
2. U.S. Pat. No. 5,645,524 Doyle. A knee support for supporting an injured knee while permitting bending and straightening of such a knee.
3. U.S. Pat. No. 5,352,190 Fischer et al. This provides for an apparatus to be used in bracing or exercising the knee joint in a manner that allows the bending of the knee joint only along a predetermined path which approximates the bending of the joint.

None of the above inventions provide the capability that the present invention provides: The protection of a load bearing and shock absorbing exoskeletal device worn around the lower limb specifically to reduce the loads and impacts on the knee joint.

The original load bearing shock absorbing device devices described under the non provisional application Ser. No. 13/555,165 with filing date of Aug. 9, 2012 confirmation number 6657, while faithfully presenting the principle of the invention i.e. the use of the overcenter linkage principle to bypass the load on the knee joint at heel strike, had some inherent design difficulties in the freedom of movement along all the axes of the limb and foot of the individual using the device.

The present design has the objective of resolving these freedoms of movement issues and additionally, provide an improved positioning of the shock absorbers with a device profile that is much less likely to interfere with the free movement of the legs as the individual participates in activities requiring a certain degree of unpredictability.

Another important feature of the present device is the increased repeatability and reproducibility of the load supporting capability of the device. This becomes increasingly critical because the differential movement of the over center linkage is very user dependent in the design versions described in the original non provisional application.

### SUMMARY OF THE PRESENT INVENTION.

The present invention provides for a load bearing shock absorbing device that is to be fastened to the limbs of the afflicted individual in the prescribed manner so that it may reduce the loads and absorb the shocks experienced by the knee joint as the individual uses the legs to walk or run or even stand still.

Essentially it comprises of a pair of strong upper struts designed to be attached to the upper limbs or thighs by means of a sleeve made of strong and flexible material incorporating specifically designed strong straps provided with Velcro or other buckle fasteners. The sleeve when wrapped around the thigh takes advantage of the somewhat conical shape of the human thigh to enable the desired load transfer from the struts to the thigh without the use of uncomfortably tight fits. The ends of the above named struts are designed to be pivotally attached to the corresponding ends of the arms of a Y shaped detail.

It is to be further noted that ends of the struts are specifically designed to enable the pivot point to be repeatably at a specific distance away from the center of rotation of the knee joint, while the two struts are positioned parallel to the axis of the thighs. The lower end of the Y shaped detail is provided with a tubular structure that can support linear bearings or allow the Y shape detail to move up and down as it is retained around a cylindrical shaft or rod specifically designed to support the friction free up and down movement. The Y shaped detail is to be fastened by specially designed straps with Velcro fasteners or buckle fasteners to the lower limbs just below the knee in a repeatable manner so as to position the pivot points in the desired position with respect to the center of rotation of the knee joint. The cylindrical shaft or bar is fixedly attached to yoke shaped detail such that it goes around the ankle and is suitable to be pivotally attached to two sliding connectors that are free to move up and down the two cooperating vertical arms of the shoe insert. The shoe inserts are made integral part of the shoe sole allowing the transfer of loads to the ground at heel strike. The shock absorbers are retained around the vertical bar in a fixed manner so that any load experienced by the shock absorbers can be conveniently transferred to the ground via the mechanism described above.

When the individuals attaches the device in the required manner to his lower limbs, the Y shaped device moves up and down the vertical bar as the individual goes from the sitting position to the standing position and starts to walk or run.

The specific design of the upper struts provides adequate magnification of the movement in a repeatable and reproducible manner so that it is possible to adjust the settings using the controls provided, to achieve the desired shock absorption and load bypass via the external shock absorbers.

The overcenter linkage principle that is being used to actuate the shock absorbers utilizing the natural movement of the limbs can best be described by reference to the diagrams in FIGS. 2 and 3. In these diagrams 701 and 704 represent the two points at which the device is strapped to the limbs: the point in the thigh where the strap is theoretically attached and the point in the shoe where the lower strut is attached. The center point of the pivot of the linkage is represented by 702 while 703 represents the center of rotation of the knee. When the individual is in the seated position, the length of the linkage 701-702-704 is greater that the length of the linkage 701-703-704 with the individual in the standing position.

Thus when the individual straps the device on in a sitting position , and then stands up, the movement of the leg forces the linkage 701-702-704 to decrease in length thereby moving the Y shaped detail downwards along the cylindrical bar. This movement is utilized to actuate the shock absorbers, until the linkage reaches the position 701-703-704 at which point the load shifts to the other leg and the limb bends allowing the shock absorber springs to return the Y shaped detail to a higher position and so on. As the individual walks or runs the movement of the limbs is sufficiently captured by the linkage to enable the shock absorbers to absorb the loads thus helping bypass the knee joint. Various configurations of shock absorbers are feasible. A shock absorber with a force response proportional to the rate of positional change of the piston is well suited for this application in order to provide greater shock absorption under more vigorous movement conditions.

One specific shape of the upper strut design that improves the repeatability of the movement and also magnifies the differential movement of the pivot point with respect to the center of knee rotation can best be described with reference to FIGS. 4 and 5. In these FIGS. 4 and 5, 101 represents the thigh of the individual, 102 represents the lower part of the limb and 105 represents the shoe. The center of rotation of the knee is represented by 105. The sleeve strapped to the thigh is represented by 40 and the upper strut 45 is designed with a small section at a 45 degree angle with the pivot point at 44. The length of this section of the strut at 45 degrees from the center of knee rotation reference point to the pivot point 44 determines the distance 110 travelled by the pivot point when the individual goes form the sitting position to the standing position.

So long as the sleeve is positioned on the thigh in a repeatable and reproducible manner, the pivot point travels a predictable distance. This enables the linkage to be designed to be responsive to small changes in angles between the upper thigh and the lower limb. The positioning of the pivot point 44 is also designed to minimize lateral loads that may be introduced in the transition from sitting to standing position.

Many design changes and improvements will become obvious to those schooled in the arts, based on these disclosures. The present descriptions are to be viewed more as illustrative of the embodied principles rather than specific design guidelines or a bill of design.

How this is to be used:

The individual puts on the specially built shoe with the load bearing insert per the design, in the seated position. He can than position the knee brace on his thigh and insert the assembly bushings on to the shoe insert verticals. At this point he can strap the lower linkage at the y detail to his calf right below the knee with strap provided by fastening snugly the straps fitted with the buckle or Velcro brand fasteners.

The sleeve detail can now be rolled snugly around the thigh and fastened tight with the two straps provided using the buckle or Velcro fasteners incorporated.

The shock absorber attachment is adjusted to transfer the required amount of movement, making sure that the device actuates at heel strike and provides the required amount of load attenuation at the knees.

As the individual walks back and forth the shock absorber position is adjusted to enable the required load reduction at the knee so that the contact between the end of the femur and the tibia is reduced resulting in immediate pain relief.

### BRIEF DESCRIPTION OF THE DRAWINGS

With reference to the attached drawings in which like reference numbers refer to like parts:

FIG. 1 shows a representation of the human knee joint with a Meniscus between the Femur and the Tibia

FIG. 2 and FIG. 3 show a representation of the “overcenter” linkage principle that is being used in this invention.

FIG. 4 and FIG. 5 show a representation of the magnified pivot point movement achieved with a specific design of the upper struts used in this invention.

FIG. 6 shows an isometric view of the preferred design that retains the freedom of the movement of the lower limb and the foot in all directions. For the sake of clarity the device is shown in isolation, as it would be with the individual getting ready to fasten it in the seated position.

FIG. 7 shows the metal load bearing insert that is made and integral part of the shoe as shown in FIG. 8

FIG. 9 shows an assembly of the lower upright fixedly attached to the lower yoke that is pivotally attached to the two bushings that are designed to slide on to the vertical uprights of the show insert.

FIG. 10 shows a shock absorber pack retained fixedly on the bracket that is fixedly retained on the lower upright depicted in FIG. 9. This FIG. 10 also shows an external return spring that may optionally be removed depending upon load requirements.

FIG. 11 shows the shock absorber supporting bracket in isolation with the optional return spring in position. FIG. 12 shows a shock absorber in an isolated isometric view.

FIG. 13 shows an isometric view of the Y detail that pivotally connects the upper struts to the lower part of the device. The strap 35 is to be used to fasten the Y detail to the upper calf in a repeatable manner. Detail 33 moves up and down the lower upright of FIG. 9. The position of the collar 34 can be adjusted to control the amount of movement that is to be transferred to the show absorbers.

FIG. 14 shows the sleeve assembly as it would be when wrapped around the individual's thigh.

FIG. 15 shows the same sleeve in flat position with the upper struts assembled with the spacers in place. FIG. 16 shows an end view of the same sleeve depicting the spacing allowed for the straps used to fasten the sleeve in place.

FIG. 17 depicts on spacer of the sleeve and FIG. 18 depicts the support detail with the countersunk screw hole also used to retain the upper struts shown in isolation in FIG. 19.

FIG. 20 depicts an isolated view of the strap used for the sleeve with the Velcro fasteners in place.

FIG. 21 depicts a view of the lower strap used to fasten the Y shaped detail to the calf just below the knee, also with the Velcro fasteners in place.

FIG. 22 depicts a isometric view of the detail used to connect the two shock absorbers used in this design. FIG. 23 shows the isometric view of the bushings pushed on to the shoe inserts and pivotally attached to the yoke depicted in FIG. 9. FIG. 24 shows an isolated view of the sleeve that is attached to the Y detail and is adjustable to control the movement transferred to the shock absorbers.

FIG. 25 shows a typical connector used in this design with one end suited for a hex drive and the other end threaded to accept a standard screw depicted in FIG. 26.

### DETAILED DESCRIPTION OF THE INVENTION

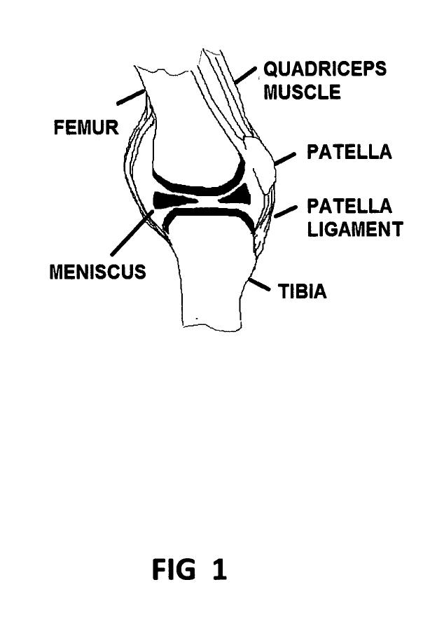

FIG. 1 shows a diagram of the lateral view of a human knee. The meniscus cartilage is represented by triangular bodies in the cross sectional view. The meniscus acts shock absorbers between the ends of the Femur and the Tibia coming together in the knee joint.

The failure of the meniscus manifests itself in tears and erosion of the meniscus body.

Normal operating conditions of the knee, whenever the individual is not at rest, require the meniscus to be supporting static and dynamic loads and as such whenever a tear in the meniscus occurs it can lead to rapid deterioration and loss of the cartilage which in turn leads to painful bone on bone contact.

It is the objective of this invention to offer an exoskeletal device incorporating suitably positioned external shock absorbers, that can be attached to the legs of the afflicted individual, which utilizing the “over center linkage” principles enable the injured knee to be “unloaded” while the load is carried by the exoskeletal structure.

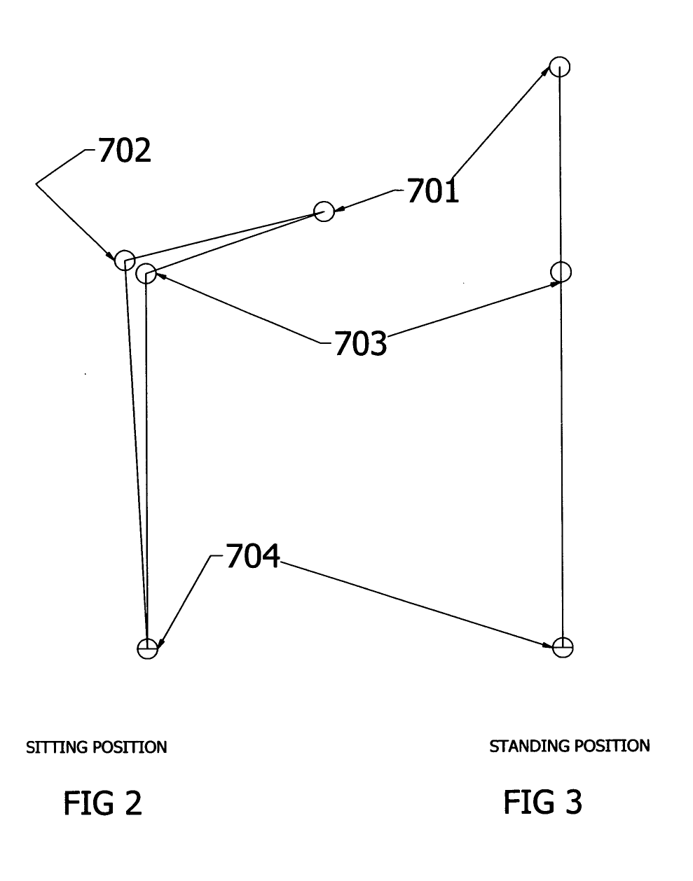

FIG. 2 and FIG. 3 show a diagram illustrating the overcenter principle which has been selected for use in the present invention and is also finds many applications in the design of clamps and buckles.

Here is an explanation of this principle in the present context. In the FIG. 2, 701 and 704 represent the points at which the device is strapped to the limbs: the point in the thigh where the sleeve is fastened and the pint in the shoe where the shoe insert is attached to the device. The center point of the clevis or the pivot point of the device is represented by 702 while 703 represents the natural canter of rotation of the knee joint. It can be seen that with the individual in the seated position the length 710-702-704 is greater than the length 701-703-704.

When the individual stands up with the linkage adequately constrained to the limbs the length of the linkage 701-702-704 is forced to decrease until it equals the length of the path in the leg 701-703-704, As shown in the FIG. 3 when the individual stands up. The shock absorbers that are made part of this linkage are designed to absorb this movement and allow the decrease in length to take place. As the shock absorbers are pushed in, they take on a part of the load that would otherwise have been supported directly by the knee joint. It is to be noted that the return springs in the shock absorber as well as any additional spring positioned in the linkage help keep the sleeve pushed up in the thigh. When the individual walks or runs the movement of the legs introduces sufficient travel in the shock absorbers to elicit a response that helps reduce the knee load. The shock absorber is to be selected based on size and packaging factors and mainly based on its ability to respond proportionately to the rate of change of the pivot point or the piston position. Vigorous and faster movements will require greater force responses to help guard the knees under shock and faster movement conditions.

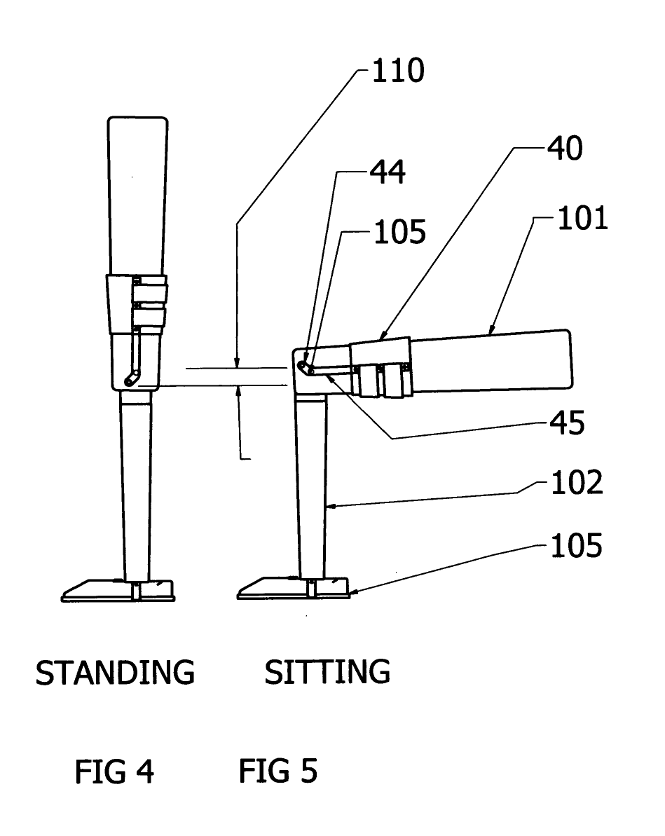

FIG. 4 and FIG. 5 show the methodology and design used to enable the overcenter principle to be used in a repeatable and reproducible manner, in the face of individual leg and knee size and shape variations.

FIG. 5 shows the individual in the sitting position with 101 representing the thigh, 102 representing the lower leg and 105 the shoe. The pivot point of the linkage is represented by 44 on the upper strut 45 attached to the sleeve 40 wrapped around the thigh 101. The strut is positioned with respect to 105 the center of rotation of the knee, such that as the individual stands up the upper strut 45 rotates along with it and the the pivot point 44 effectively travels a distance represented by 110. As long as this movement is repeatably available to actuate the shock absorbers, the device can be adjusted to provide the desired force response in a reproducible and repeatable manner from day to day and from individual to individual.

Another aspect of this design is that when the upper struts are aligned with the axis of the thigh and the pivot point is positioned as described with respect to the center of rotation of the knee, the lateral forces experienced by the device are reduced to an acceptable level.

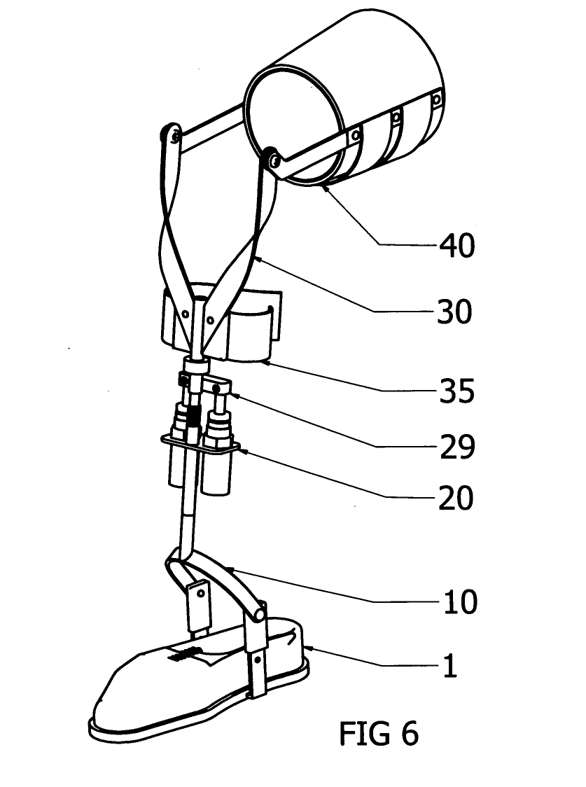

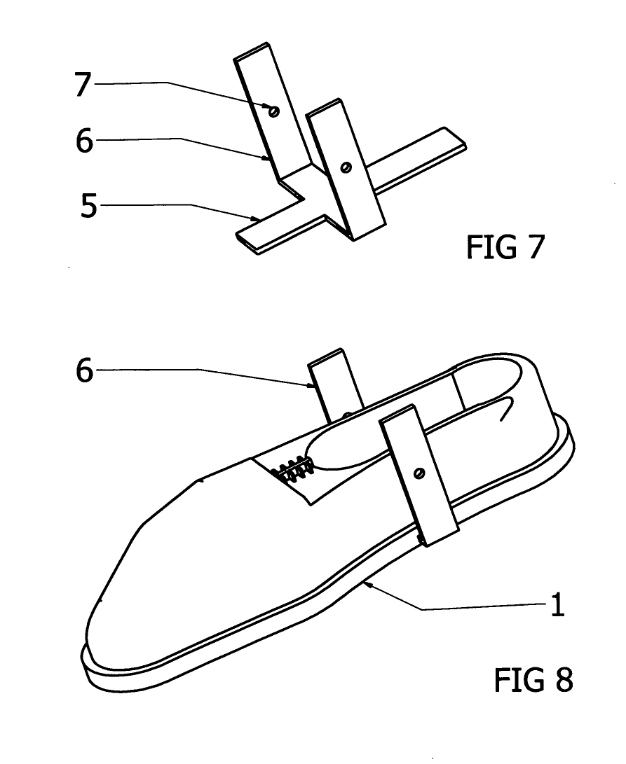

FIG. 6 on page 4/14 shows an isolated isometric view of the preferred exoskeletal, load bearing, shock absorbing knee brace as it would be with the individual in the seated position.

The person who is to wear the brace is not shown in the view but it can be seen that the individual would lace on the shoes, engage the shoe insert verticals with the bushings pivotally mounted on the yoke 10. The individual would proceed to snug tight fastener 35 around his calf just below the knee. At this point he would roll the sleeve around his thigh and wrap it snug tight with the two straps provided with Velcro fasteners. The adjustments available to the individual will be described at a later stage, so at this point the individual knows that the device has been adjusted to his particular requirements.

It is noted that has he stands up the Y shaped detail, pivotally connected to the upper struts in sleeve 40, slides down on shaft 13 that is made an integral part of the yoke 10 assembly. The Y shaped detail is designed to rotate freely and move up and down the shaft 13 so as not to restrict the angular movement of the foot of the individual.

Depending upon the adjustments, at some point before the individual is fully standing, the Y shaped detail assembly starts making contact with the shock absorber pack, which is positioned in front of the tibia minimizing the potential for interference with the other leg or the even the device on the other leg, should the individual require a device on each of his legs.

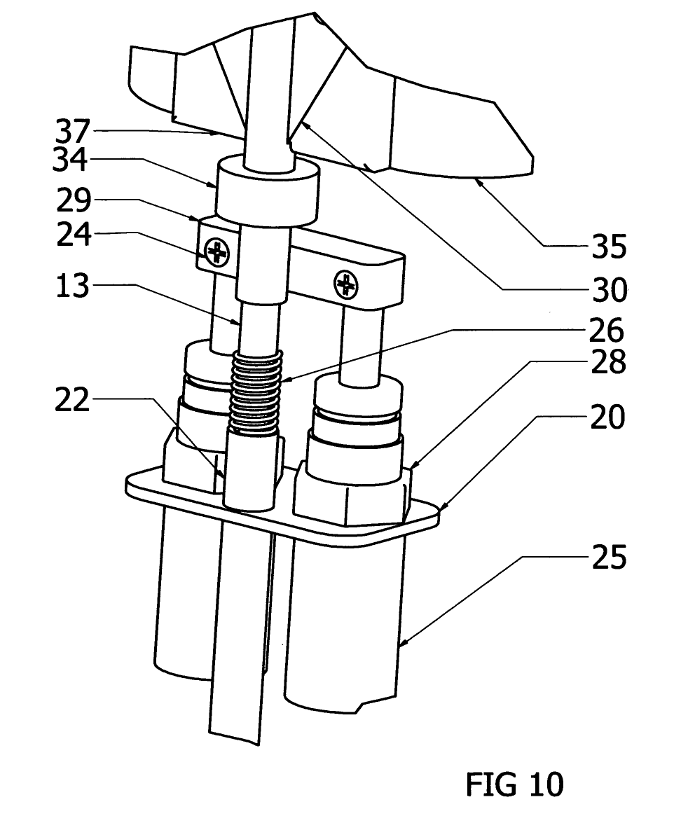

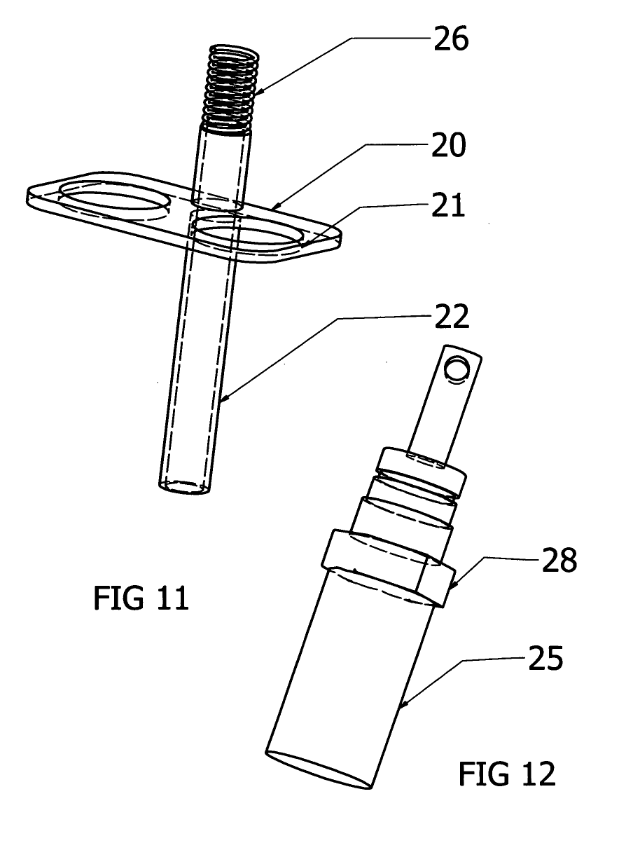

With reference to FIG. 10 on page 7/14 we can see here the shock absorber pack fixedly mounted on bracket 20 such that the nut 28 and the threads on the body 25 of the shock absorber can be used to adjust the position as required. The detailed view of the shock absorber 25 is also shown in FIG. 12 on page 8/14. This page also shows FIG. 11 with the spring 26 and the bracket 20 constructed with the tube 22 with dimensions that permit a close fit with shaft 13 mentioned earlier. The shock in this design are connected in a load bearing manner by a bar 29 held in place with screws 24. Even though these shock absorbers are provided with internal return springs an additional external spring 26 is provided to help keep the sleeve pushed up against the thigh and with additional static load support.

FIG. 10 also shows the shaft 13 on which the shock absorber pack is positioned in a fixed manner so that any force exerted on detail 29 can be transferred to the ground via detail 13, the yoke 10 and the shoe 1. Also in FIG. 10 the lower end of the Y shaped detail 30 can be seen coaxial to the bar 13 and free to move up and down within the constraints provided. The collar 34 can be moved and locked in position on the lower end of the Y shaped detail, and it is this collar that engages the bar 29 as the individual walks or runs and transfers the load.

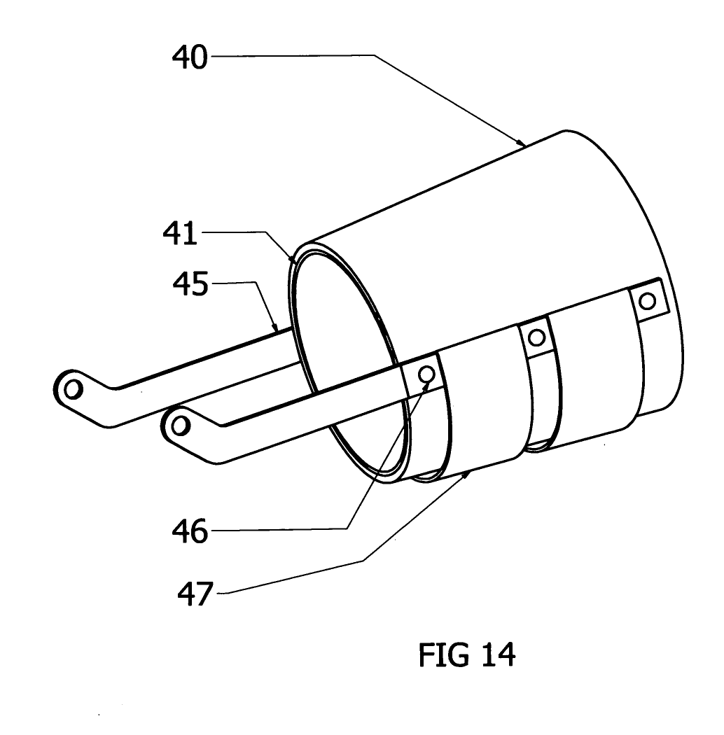

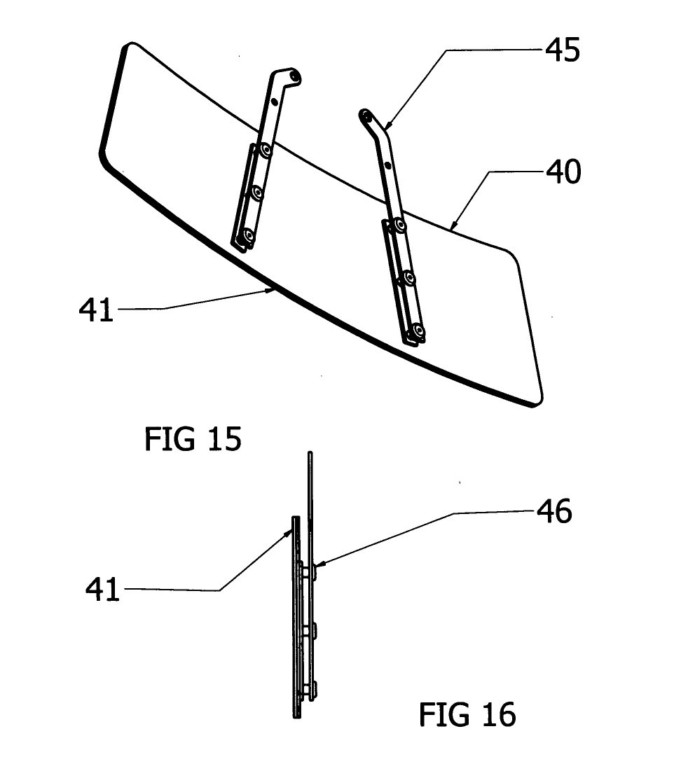

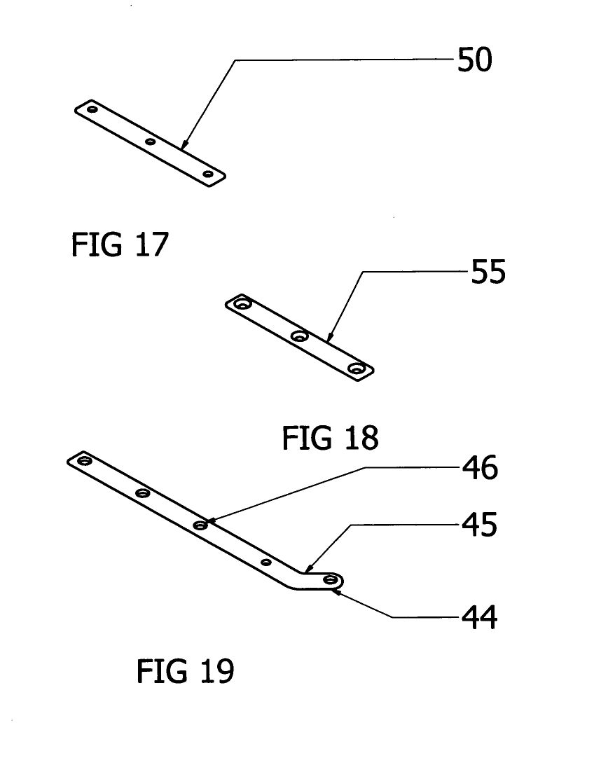

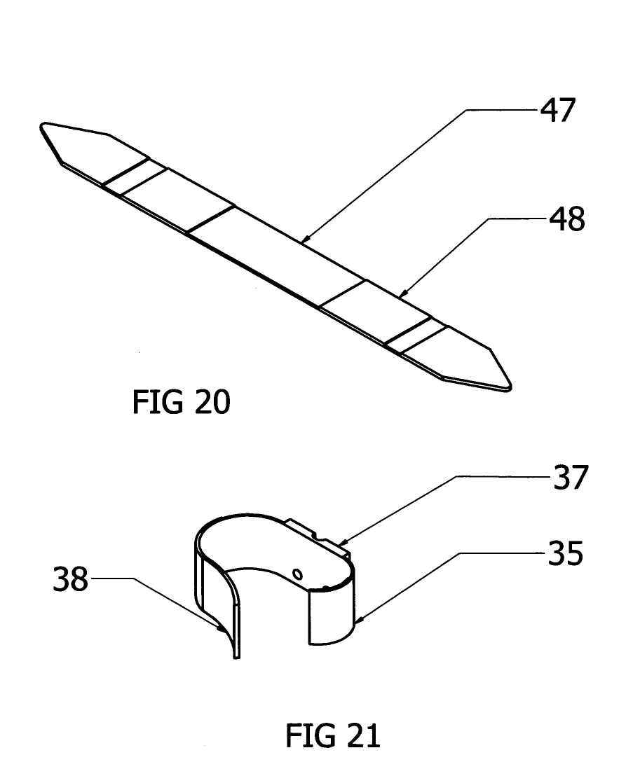

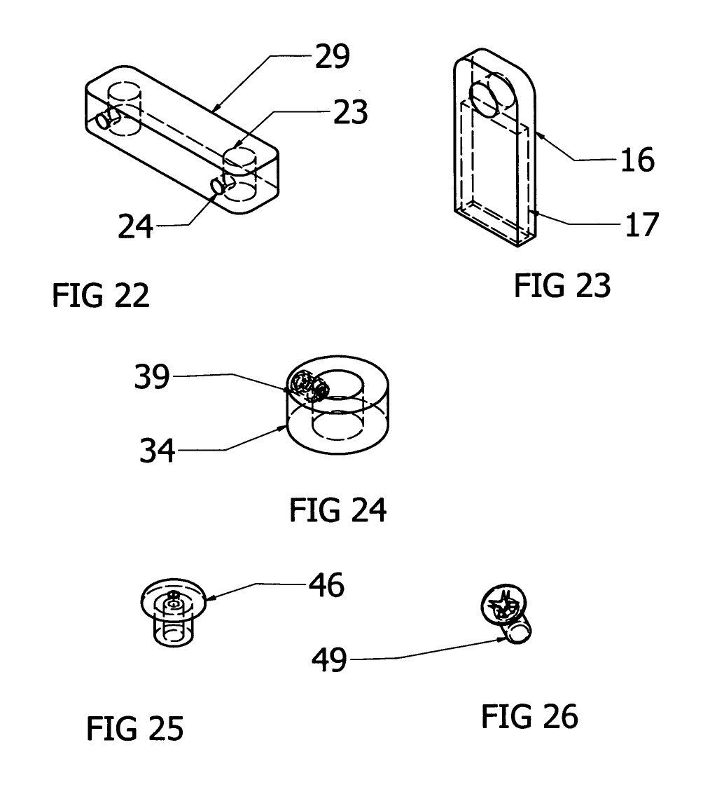

FIGS. 14 through 20 and 25-26 depict the thigh sleeve and its components. FIG. 15 shows the construction technique of one preferred design. Detail 40 is the main sleeve detail made out of a strong yet flexible material and 41 is the inner liner that comes in contact with the skin and accordingly made of non allergenic strong and soft material designed for user comfort. FIG. 15 also depicts the construction technique used to attach the upper struts 45 also shown in detail in FIG. 19. Connector nut 46 shown in FIG. 25 and screws 49 shown in FIG. 26 are used to assemble the sleeve and the upper struts 45 using the spacers 50 and 55 as shown in the end view presented in FIG. 16 so that the straps 47 built with Velcro fasteners 48 can be used to retain the rolled up sleeve as shown in FIG. 14.

Turning our attention to FIG. 7 and FIG. 8 we see the shoe insert in FIG. 7 designed to support the knee brace on the verticals 6. The verticals are also provided with threaded holes for use with additional support straps in case those are desired for extra active applications. FIG. 8 depicts a shoe with the insert 5 made an integral part such that only the verticals 6 are visible and available to support the knee brace.

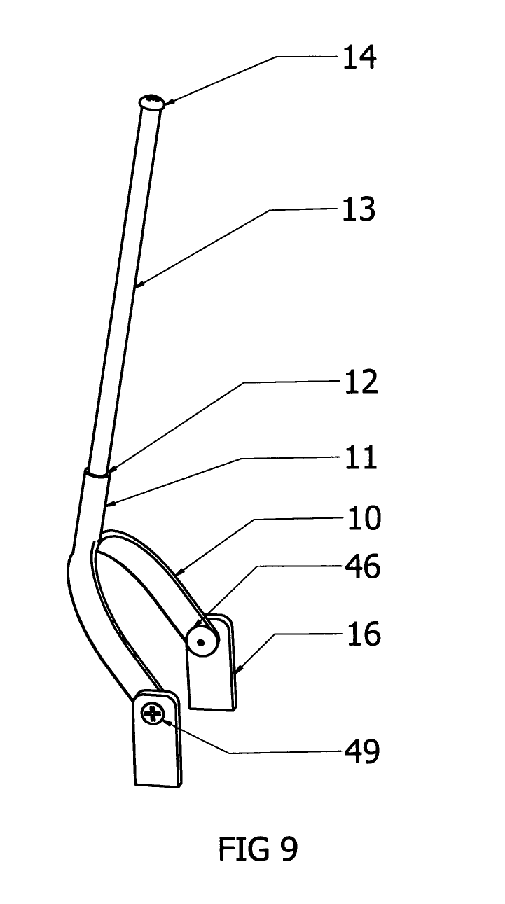

FIG. 9 on page 6/14 shows a subassembly of the yoke detail 10, with the bar 13 made integral by welding. The bushings 16 designed to closely fit over the verticals of the shoe insert described above are pivotally attached to the ends of the yoke using the connector 46 with the fastener 49. The design of this subassembly supports the objective of providing freedom of movement along all axes. The shoulder 12 is provided to support the shock absorber pack, and the fastener 14 is to be used to retain the sub assembly in place.

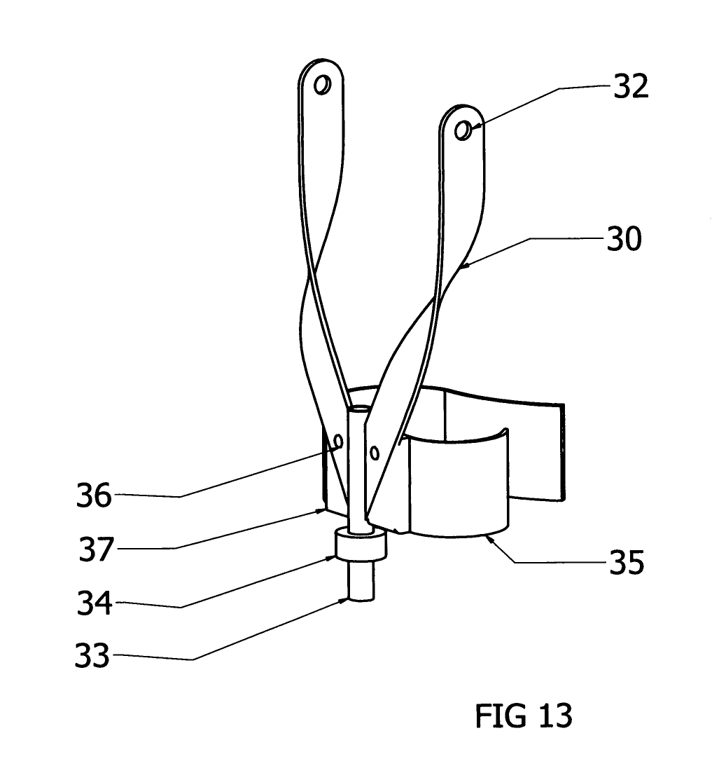

FIG. 13 on page 9/14 depicts an isometric view of the Y shaped subassembly that is to be pivotally connected through holes 32 using fasteners 46 and 49 previously described. The lower end of the Y is fixedly connected by welding to a tubular piece designed to freely move up and down the shaft 13 described above. The bushing detail 34 can be locked in a given position on the tubular end of the Y, as required, by using the set screw 39 shown in FIG. 24 on page 14/14.

The detail 34 makes contact with the shock absorber pack and provides the required shock absorber actuation movement. As the Y slides up and down the detail 13 shaft, the end comes in contact with the spring 26. The dimensions of these details are such that the spring bottoms up before the shock absorber reaches the bottom of the stroke. This can also help maximize load transfer through the brace bypassing the knee joint at the bottom of the stroke.

The strap 35 provided connected to the Y with the spacer 37 using the holes threaded 36 and fasteners 49 is designed to allow the user a comfortable, repetitively accurate connection. This strap is also shown in isolation in FIG. 21 where the Velcro 38 used for fastening can be seen.

What is being claimed:

1. An exoskeletal load bearing shock absorbing knee brace designed to support the knee principally by reducing the load on the knee and absorbing peak shock loads by use of a pair of specifically designed external shock absorbers held in working position by an upper sleeve assembly consisting of a pair of struts held parallel to the upper leg by the sleeve which is firmly cuffed to the upper part of the leg or the thigh and a lower strut assembly held in place parallel to the lower limb by cuffing to the calf and supported in a load bearing pivotal manner by a load bearing shoe sole insert which is made integral part of the shoe of the individual using said knee brace.

2. The exoskeletal load bearing and shock absorbing knee brace of claim one wherein the upper strut assembly and the lower strut assembly are pivotally connected to each other in such a way that with the individual using the brace in the seated position, the pivot point is at a certain distance away from the natural pivot point of the knee of the individual using the brace, and such that when the individual stands up, the distance differential between the two pivot points is to be overcome by the displacement of the shock absorber piston and the piston rod, and furthermore any relative angular movement between the upper and lower limbs is to cause a corresponding actuating displacement of the shock absorber piston thereby producing the associated shock absorber load response, said shock absorber being designed to respond to minute changes in displacement of the piston.

3. The exoskeletal load bearing and shock absorbing knee brace of claim one wherein the upper strut assembly and the lower strut assembly are dimensionally matched to the leg of the individual using said brace, so as provide the distance differential between the two pivot points required to activate the shock absorbers.

4. The exoskeletal load bearing and shock absorbing knee brace of claim one wherein the shock absorbers are designed to optimize the actuation requirements and the corresponding load response so that the load on the knees of the user is reduced as required.

5. The exoskeletal load bearing shock absorbing knee brace of claim two wherein the shock absorbers are provide with external return springs or preferably internal return springs that are capable of returning the shock absorber piston to the top of the stroke under no load conditions and also capable of pushing the connected upper sleeve snugly up against the upper thigh of the individual taking advantage of the conical shape of the lower part of the human thigh.

6. The exoskeletal load bearing and shock absorbing knee brace of claim one wherein the upper strut are supported in a load bearing capable manner by the upper sleeve designed to fit the conical shaped human thigh, and held in place snuggly by straps thus enabling the transfer of load directly, bypassing the knee joint.

7. The exoskeletal load bearing and shock absorbing knee brace of claim one wherein the upper strut is designed to provide a repetitive and reproducible differential movement based on a an exact distance between the center of rotation of the knee and position of the pivot point at which it is connected to the lower strut assembly.

8. The exoskeletal load bearing shock absorbing knee brace of claim two wherein the lower strut assembly consists of a main shaft fixedly attached to a yoke shaped detail that is pivotally supported on two bushings that slide onto the verticals of the shoe insert in manner that permits transmission of loads to the shoe sole, while retaining the freedom of movement of the lower leg and foot along all the axes.

9. The exoskeletal load bearing shock absorbing knee brace of claim 7 wherein the vertical shaft supports the shock absorber pack in a fixed manner away from any contact with the lower leg of the individual using it.

10. The exoskeletal load bearing shock absorbing knee brace of claim 7 wherein the vertical shaft also supports the Y shaped detail in a coaxial yet capable of freely moving linearly and rotatably, making possible the retention of the freedom of movement of the lower leg in all directions.

11. The exoskeletal load bearing shock absorbing knee brace of claim 7 wherein the vertical shaft also supports the Y shaped detail which is also pivotally connected to the upper struts of claim 6 and is fastened snuggly to the calf of the individual using the knee brace, using straps provided with Velcro or buckle fasteners so that the pivot points are positioned in a repetitive and reproducible manner with respect to the center of rotation of the knee.

12. The exoskeletal load bearing shock absorbing knee brace of claim seven wherein the loose fit between the bushing pivotally connected to the yoke and the verticals of the show is such that it allows a quick connect and disconnect as well as it allows the two details to move freely, yet is sufficient to maintain the connection in place at all times until disconnected by the individual using the brace.

13. The exoskeletal load bearing shock absorbing knee brace of claim seven wherein the load bearing shoe insert is made an integral part of the sole of the shoe such that it can transfer evenly to the ground the load being imposed on it by the lower strut assembly of the knee brace.

14. The exoskeletal load bearing shock absorbing knee brace of claim seven wherein the quick disconnect feature for attachment of the brace to the limbs may additionally include a strap made out of flexible yet strong and durable material fitted with Velcro brand loop and hook fasteners or even a strap fitted with an overcenter buckle attached to the verticals of the shoe insert.
